(19) United States
(12) Patent Application Publication
(10) Pub. No.: US 2009/0210039 A1
(43) Pub. Date: Aug. 20, 2009

(54) Prosthetic system for therapeutic optical activation and silencing of genetically-targeted neurons

(76) Inventors: Edward S. Boyden, Cambridge, MA (US); Jacob G. Bernstein, Cambridge, MA (US); Michael Henninger, Cambridge, MA (US)

Correspondence Address: NORMA E HENDERSON;HENDERSON PATENT LAW, 13 JEFFERSON DR, LONDONDERRY, NH, 03053, US

(21) Appl. No.: 12/355,745

(22) Filed: Jan. 16, 2009

Related U.S. Application Data
(63) Continuation-in-part of application No. 12/118,673, filed on May 9, 2008.
(60) Provisional application No. 61/021,612, filed on Jan. 16, 2008.
(60) Provisional application No. 60/917,055, filed on May 9, 2007.

(51) Int. Cl. A61N 5/067; A61N 5/06

(52) U.S. Cl. 607/89; 607/88

(57) ABSTRACT

An optical prosthesis that permits control of neural circuits comprises a probe having a set of light sources, drive circuit connections connected to each light source, a housing surrounding the light sources and drive circuit connections, and drive circuitry for driving and controlling the probe. The drive circuit connections and drive circuitry may optionally provide for wireless communication. The light sources may be light-emitting diodes, lasers, or other suitable sources. The device may optionally include sensors for monitoring the target cells. The present invention is also a multi-dimensional array of probes, each probe having a set of light sources, drive circuit connections connected to each light source, a housing surrounding the light sources and the drive circuit connections, drive circuitry for driving and controlling the probes, and supporting hardware that holds the probes in position with respect to each other and the target cells.

### RELATED APPLICATIONS

This application claims the benefit of U.S. Provisional Application Ser. No. 61/021,612, filed Jan. 16, 2008, the entire disclosure of which is herein incorporated by reference.

This application is also a continuation-in-part of co-pending U.S. patent application Ser. No. 12/118,673, filed May 9, 2008, which claims the benefit of U.S. Provisional Application Ser. No. 60/917,055, filed May 9, 2007, the entire disclosures of which are herein incorporated by reference.

### STATEMENT REGARDING FEDERALLY SPONSORED RESEARCH OR DEVELOPMENT

This invention was made with U.S. government support under Grant Number DP2OD002002, awarded by the National Institutes of Health (NIH). The government has certain rights in this invention.

### FIELD OF THE TECHNOLOGY

The present invention relates to methods and devices for control of cell function and, in particular, to prosthetic devices for optical control of cells.

### BACKGROUND

Many neural disorders are associated with aberrant activity in specific cell types or neural projection pathways embedded within the densely-wired, heterogeneous matter of the brain. Many diseases of the human brain and nervous system are related to the dysfunction of specific neuron types that undergo pathological changes in number, excitability, anatomy, or synaptic connectivity. These changes lead, via altered neural circuit activity, to the perceptual, cognitive, emotional, and motor deficits associated with various neurological and psychiatric illnesses. For example, temporal lobe epilepsy is associated with increased excitability and connectivity of specific excitatory neurons [C. Bernard, A. Anderson, A. Becker et al., Science 305 (5683), 532 (2004); E. R. Sanabria, H. Su, and Y. Yaari, J Physiol 532 (Pt 1), 205 (2001); L. R. Shao and F. E. Dudek, J Neurophysiol 92 (3), 1366 (2004); C. R. Houser, J. E. Miyashiro, B. E. Swartz et al., J Neurosci 10 (1), 267 (1990)] and the loss of specific kinds of inhibitory interneurons [P. S. Buckmaster and F. E. Dudek, J Comp Neurol 385 (3), 385 (1997)] in the hippocampus, whereas schizophrenia is associated with atrophy of a specific kind of inhibitory neuron in the prefrontal cortex [D. A. Lewis, T. Hashimoto, and D. W. Volk, Nat Rev Neurosci 6 (4), 312 (2005)].

These cell classes, or other cell types within the affected neural circuits, may possibly serve as novel and powerful clinical targets. An ideal therapy would permit correction of activity just in specific target neurons, while leaving other neurons unaltered. By activating and silencing specific cell classes, perhaps even in an adaptive way that depends upon the state of the neural circuit, it may be possible to devise efficacious, side effect-free treatments for a multitude of neurological and psychiatric diseases. For example, a disease in which a specific kind of neuron has become pathologically overexcitable, such as is the case in epilepsy, may be treatable via cell-specific neural silencing, whereas a disease in which a specific neuron has become underactive (as in the case of schizophrenia) may be treatable via cell-specific neural activation. However, until recently, tools precise enough to perform this kind of neural-circuit level activity sculpting have not existed.

Neurologists and psychiatrists have altered neural activity in the brain via the use of non cell-type specific electromagnetic methods (e.g., deep brain stimulation (DBS) and transcranial magnetic stimulation (TMS)), showing that stimulating a single bulk chunk of neural tissue within the brain can reliably, although often only partially, alleviate symptoms of disorders as diverse as depression [M. S. George, E. M. Wassermann, W. A. Williams et al., Neuroreport 6 (14), 1853 (1995); A. Pascual-Leone, B. Rubio, F. Pallardo et al., Lancet 348 (9022), 233 (1996); H. S. Mayberg, A. M. Lozano, V. Voon et al., Neuron 45 (5), 651 (2005)], epilepsy [D. A. Groves and V. J. Brown, Neuroscience and biobehavioral reviews 29 (3), 493 (2005); K. N. Fountas, J. R. Smith, A. M. Murro et al., Stereotactic and functional neurosurgery 83 (4), 153 (2005); M. Morrell, Current opinion in neurology 19 (2), 164 (2006)], chronic pain [D. Rasche, P. C. Rinaldi, R. F. Young et al., Neurosurgical focus 21 (6), E8 (2006); R. R. Tasker and O. Vilela Filho, Stereotactic and functional neurosurgery 65 (1-4), 122 (1995)], Parkinson's disease [A. L. Benabid, P. Pollak, A. Louveau et al., Applied neurophysiology 50 (1-6), 344 (1987); R. P. Iacono, R. R. Lonser, G. Maeda et al., Acta neurochirurgica 137 (1-2), 106 (1995); R. Pahwa, S. Wilkinson, D. Smith et al., Neurology 49 (1), 249 (1997); R. Kumar, A. M. Lozano, Y. J. Kim et al., Neurology 51 (3), 850 (1998); L. Lopiano, M. Rizzone, B. Bergamasco et al., Neurology 56 (4), 552 (2001); A. L. Benabid, P. P. Krack, A. Benazzouz et al., Neurology 55 (12 Suppl 6), S40 (2000)], and cluster headache [M. Leone, Lancet Neurol 5 (10), 873 (2006)]. These stimulation technologies overcome some of the issues associated with other methods, such as the irreversibility of surgical ablation of brain tissue [W. Penfield and T. Rasmussen, The Cerebral Cortex of Man: A Clinical Study of Localization of Function. (Macmillian 1950)] and the widespread side effects associated with many drugs [P. I. Rosebush and M. F. Mazurek, Neurology 52 (4), 782 (1999); J. L. Herranz, J. A. Armijo, and R. Arteaga, Epilepsia 29 (6), 794 (1988); S. L. McElroy, P. E. Keck, Jr., and L. M. Friedman, The Journal of clinical psychiatry 56 Suppl 2, 49 (1995)]. However, systematic principles of how to control aberrant activity in neural circuits have not been described, in part because of the nonspecific nature of electrical and magnetic stimulation. Such fields cannot be focused at a distance due to their fundamental physical properties, and cannot be targeted to specific cell types within a volume of tissue due to the relative nonselectivity of the action of such fields upon neurons.

The ability to optically activate or inactivate genetically-specified excitable target cells, such as central nervous system neurons, glia, peripheral neurons, skeletal muscle, smooth muscle, cardiac muscle, pancreatic islet cells, thymus cells, immune cells, or other excitable cells, embedded in intact tissue, such as brain, peripheral nervous system, muscle, and skin, would enable radical new treatments for many disorders (e.g., neuropathic pain, Parkinson's disease, epilepsy, diabetes, and other diseases). Ideally, for each disease, which neural cell targets offer the greatest therapeutic potential and what patterns of activity must be imposed in order to optimally correct the pathological state may be identified. In this way, principles of neural control can be identified that permit optimal resculpting of the aberrant activity in the brain that contributes to illness. It might further be possible to use optical neural control technologies directly as a therapy, sensitizing specific targets in the human brain to light and then activating, silencing, or reshaping their activities as required.

Molecular genetic methods for making cells, such as neurons, sensitive to being activated (e.g., depolarized) or inactivated (e.g., hyperpolarized) by light have been previously developed. For example, in X. Han and E. S. Boyden, “Multiple-color optical activation, silencing, and desynchronization of neural activity, with single-spike temporal resolution,” PLoS ONE 2, e299 (2007) and E. S. Boyden, F. Zhang, E. Bamberg et al., Nat Neurosci 8 (9), 1263 (2005), tools are described that permits specific genetically-defined excitable cells, such as neurons of different classes, to be activated and silenced with different colors of light. These tools center around a set of naturally-occurring membrane proteins that, when exposed to light of the appropriate wavelength, move ions from one side of the plasma membrane to the other. The naturally-occurring light-activated proteins channelrhodopsin-2 (ChR2) and halorhodopsin (Halo NpHR) can, when genetically expressed in neurons, enable them to be safely, precisely, and reversibly activated and silenced by pulses of blue and yellow light, respectively. FIGS. 1A-G show that the light-activated cation channel channelrhodopsin-2 (abbreviated ChR2, from the green algae C. reinhardtii), when expressed in neurons, makes neurons optically activatable by millisecond-timescale pulses of blue light (FIGS. 1A, 1B); the light-activated chloride pump halorhodopsin (abbreviated Halo or NpHR, from the archaebacterium N. pharaonis) similarly makes neurons electrically silenceable by pulses of yellow light (FIGS. 1C, 1D); and together these molecular tools, when expressed in an appropriate ratio, can enable bi-directional control of neural activity by pulses of blue and yellow light (FIGS. 1E-G), enabling spike-level neural activity control. These molecules can be targeted to neurons by any of the means available to modern biotechnology, including the use of viruses or transgenic methods.

As shown in FIGS. 1A-G, neuron-expressing channelrhodopsin-2 is fused to mCherry (FIG. 1A; bar, 20 μm) and halorhodopsin is fused to GFP (FIG. 1C). The overlay is shown in FIG. 1E. In FIG. 1B, Poisson trains of spikes 105, 110 are elicited by pulses of blue light 115, 120 in two different neurons. In FIG. 1D, light-driven spike blockade 130 is depicted for a representative hippocampal neuron, and another light-driven spike blockade 135 is shown for a population of neurons (n=7). Neuronal firing was induced by pulsed somatic current injection (300 pA, 4 ms). Hyperpolarization was induced by periods of yellow light 140. As seen for spike blockade 135, yellow light 140 drives Halo to block neuron spiking, leaving spikes elicited during periods of darkness intact. In FIG. 1F, action spectrum 150 for ChR2 overlaid with absorption spectrum 155 for N. pharaonis halorhodopsin. FIG. 1G depicts hyperpolarization and depolarization events 160 induced in a representative neuron by a Poisson train of alternating pulses (10 ms) of yellow 170 and blue 175 light.

While these molecular genetic methods make cells, such as neurons, sensitive to being activated or inactivated by light, methods are for delivering light to precise locations in intact tissues are still required. In co-pending U.S. patent application Ser. No. 12/118,673, published as U.S. Pat. App. Pub. No. 2008/0306576, several first-generation devices are described for accomplishing this, including sets of light sources (e.g., LEDs, lasers) coupled to optical fibers whose ends deliver light to specified groups of target cells within tissue, sets of light sources in hypodermic cannulas that can deliver light locally to specified groups of target cells within tissue, and sets of light sources attached to nerve cuff holding devices that stably bring the light sources into close proximity to a group of target nerve cells. In conjunction with the sets of light sources, control and power electronics enable battery-powered, wearable, fully implantable, wirelessly-operated, and/or remotely-powered versions of the electronics to drive the light sources, thus enabling the use of the devices as prosthetics. While simple and flexible, the laser setup of this approach can be expensive, bulky, and consume a lot of power. Furthermore, since it requires optical fibers to be inserted into the brain that are simultaneously connected to a heavy, fixed laser, use of such a system constrains the movement of animals and increases the risk of tangling or breakage of optical fibers. These problems worsen if insertion of multiple fibers is desired. In addition, optical fibers emit light only at their end, meaning that an optical fiber damages a significant volume of brain tissue, compared to the amount of brain tissue that is illuminated.

### SUMMARY

The present invention is an optical prosthesis that can be used to directly remedy aberrant activity in corrupted human brain circuits by controlling neural circuits. It is a scalable, fully-implantable optical prosthetic capable of delivering light of appropriate intensity and wavelength to targeted neurons at arbitrary 3-D locations within the brain, enabling activation and silencing of specific neuron types at multiple locations. The device can be implanted in the brains of animals in order to reveal principles of neural control, and also serves as a prototype for optical prosthetics that may be practical for human use.

In one aspect, the present invention comprises a set of light sources with optional accessory hardware for guiding light, supporting hardware to hold members of the set of light sources with respect to each other, with respect to the target cells, and perhaps with respect to external structures, and control and drive electronics that provide regulated power to the light sources, communicate data, stimulation protocols, and algorithms to and from the outside world, and optionally monitor target cell state.

In one embodiment, the present invention is a prosthetic device for optical control of target cells, comprising a probe having a set of light sources, drive circuit connections connected to each light source, a housing surrounding the light sources and the drive circuit connections, and drive circuitry for driving and controlling the probe. The drive circuit connections and drive circuitry may optionally provide for wireless communication. The light sources may be light-emitting diodes, lasers, or any other suitable source known in the art. The housing may be a glass capillary tube. The prosthetic device may optionally include sensors for monitoring the target cells.

In another embodiment, the present invention is a prosthetic device for optical control of target cells, comprising an array of probes, each probe having a set of light sources, drive circuit connections connected to each light source, a housing surrounding the light sources and the drive circuit connections, drive circuitry for driving and controlling the probes, and supporting hardware that holds the probes in position with respect to each other and the target cells. The array of probes may be two- or three-dimensional.

### BRIEF DESCRIPTION OF THE DRAWINGS

Other aspects, advantages and novel features of the invention will become more apparent from the following detailed description of the invention when considered in conjunction with the accompanying drawings wherein:

FIG. 1A is a photograph depicting neurons optically activated through the light-activated cation channel channelrhodopsin-2 by millisecond-timescale pulses of blue light;

FIG. 1B depicts Poisson trains of spikes elicited by pulses of blue light in two different neurons;

FIG. 1C is a photograph depicting neurons electrically silenced through light-activated chloride pump halorhodopsin by pulses of yellow light;

FIG. 1D depicts light-driven spike blockades for a representative hippocampal neuron and for a population of neurons, with hyperpolarization induced by periods of yellow light;

FIG. 1E is a photograph depicting bi-directional control of neural activity by pulses of blue and yellow light, enabling spike-level neural activity control;

FIG. 1F is a graph depicting an action spectrum for channelrhodopsin-2 overlaid with an absorption spectrum for N. pharaonis halorhodopsin;

FIG. 1G is depicts hyperpolarization and depolarization events induced in a representative neuron by a Poisson train of alternating pulses of yellow and blue light;

FIG. 2 is an exemplary embodiment of a 3D LED array for enabling activation and silencing of activity in targeted brain regions, according to one aspect of the present invention;

FIG. 3A is an expanded view of an exemplary embodiment of a 1-D probe useful in constructing a 3D array, according to one aspect of the present invention;

FIG. 3B a photograph of a prototype implementation of the ID probe of FIG. 3A, according to one aspect of the present invention;

FIG. 3C is an exemplary embodiment of an LED driver circuit useful with a 3D array, according to one aspect of the present invention;

FIG. 4 is schematic of an exemplary setup for focusing blue and yellow light down a fiber to be implanted inside the head, for bi-directional control of a single kind of neuron at a single site within the brain, according to one aspect of the present invention;

FIGS. 5A and 5B are Monte Carlo simulations of how blue and yellow photons, respectively, travel through the brain from LEDs, according to one aspect of the present invention;

FIG. 6 is a photograph of neurons in the mouse brain expressing Halo-GFP under the CaMKII promoter, which preferentially labels excitatory neurons; and

FIG. 7 is an experimentally-produced raster plot indicating occurrences of spikes elicited from an excitatory neuron expressing ChR2 in a monkey cortex in response to a brief train of blue light stimulation.

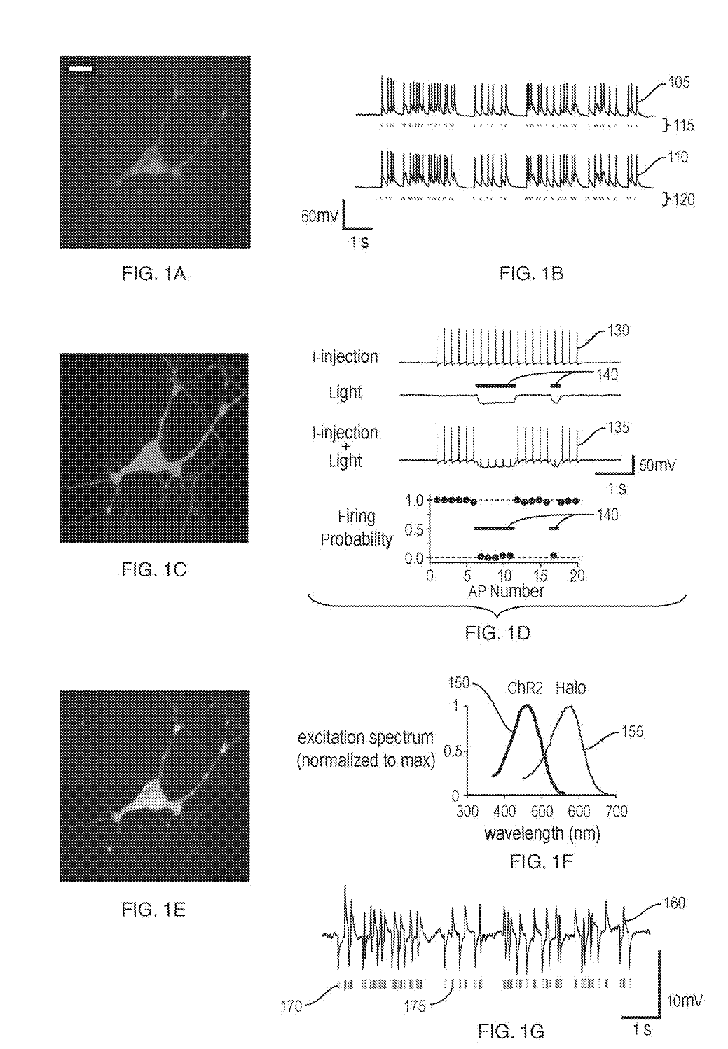

### DETAILED DESCRIPTION

The present invention comprises several parts: a set of light sources (such as, but not limited to, LEDs or lasers) with optional accessory hardware for guiding light, supporting hardware to hold members of the set of light sources with respect to each other, with respect to the target cells such as, but not limited to, brain, glia, peripheral nerve, skeletal muscle, smooth muscle, cardiac muscle, pancreatic islet cells, thymus cells, or other excitable cells, embedded in the tissue (such as, but not limited to, brain, peripheral nervous system, muscle, skin, pancreas, and heart), and perhaps held firm with respect to external structures (such as, but not limited to, skull, skeleton, muscle, or skin), and control and drive electronics that monitor target cell state, provide regulated power to the light sources, communicate data, stimulation protocols, and algorithms to and from the outside world, and/or may be remotely powered by external electromagnetic fields or other kinds of wireless energy. A set of light sources is needed because, since tissues are highly scattering, in many cases no one light source will be able to illuminate all the target cells in the entire desired target area. Each individual light source must receive electrical power and deliver light locally to its target cells.

In a preferred embodiment, a 3-D array of LEDs capable of targeting arbitrary brain structures enables activation, silencing, and the resculpting of activity throughout multiple brain regions. This 3D array provides a probe that is inexpensive, compact, power-efficient, self-contained, and capable of exerting optical control over a large region of brain tissue while causing a minimum of brain damage. The present invention employs such 3-D arrays of compact, inexpensive, long-lasting, and bright light-emitting diodes (LEDs), capable of targeting arbitrary brain regions for activation or silencing. Unlike an optical fiber, which can only deliver light to a single point in the brain per penetrating device, a linear array of LEDs inserted into the brain can deliver light to points in the brain up and down the probe. In the preferred embodiment, the device is compact (human DBS electrodes are <1.5 mm wide), is capable of delivering bright light into deep brain structures, and does not heat the brain beyond an acceptable limit (typically, 1° C.). One efficient way to implement a 3-D array of LEDs is to create a 2-D array of 1-D probes.

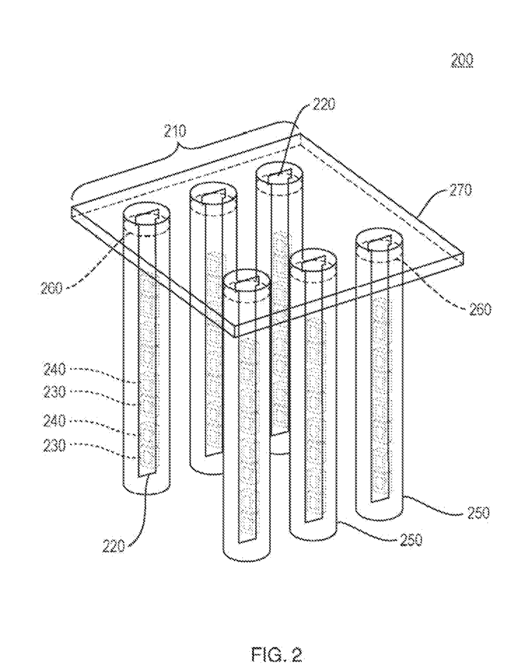

FIG. 2 is an exemplary embodiment of a device that enables 3-D arrays of yellow and blue LEDs to activate, silence, and resculpt neural activity in arbitrary 3-D patterns. As shown in FIG. 2, device 200 is a 2-D array 210 of 1-D LED-bearing probes 220, each of which has alternating blue 230 and yellow 240 LEDs going down its length. The 1-D probes 220 are placed in glass capillaries 250 to prevent brain heating. The capillaries are then arranged into 2-D array 220 by placing them in form-fitting holes 260 within plastic plate 270. Each 1-D probe projects vertically down into the brain. Glass capillary 250 electrically and thermally isolates the LEDs from the brain; the heat is instead wicked up the 1-D probe to cool the LEDs. The 2-D array 210 is assembled by placing multiple glass capillaries 250 containing 1-D probes 210 into cylindrical holes 260 in plastic plate 270 and then sealing capillaries 250 to plate 270 using epoxy. Such an array of probes then enables optical control of arbitrary 3-D sets of neurons distributed in multiple regions.

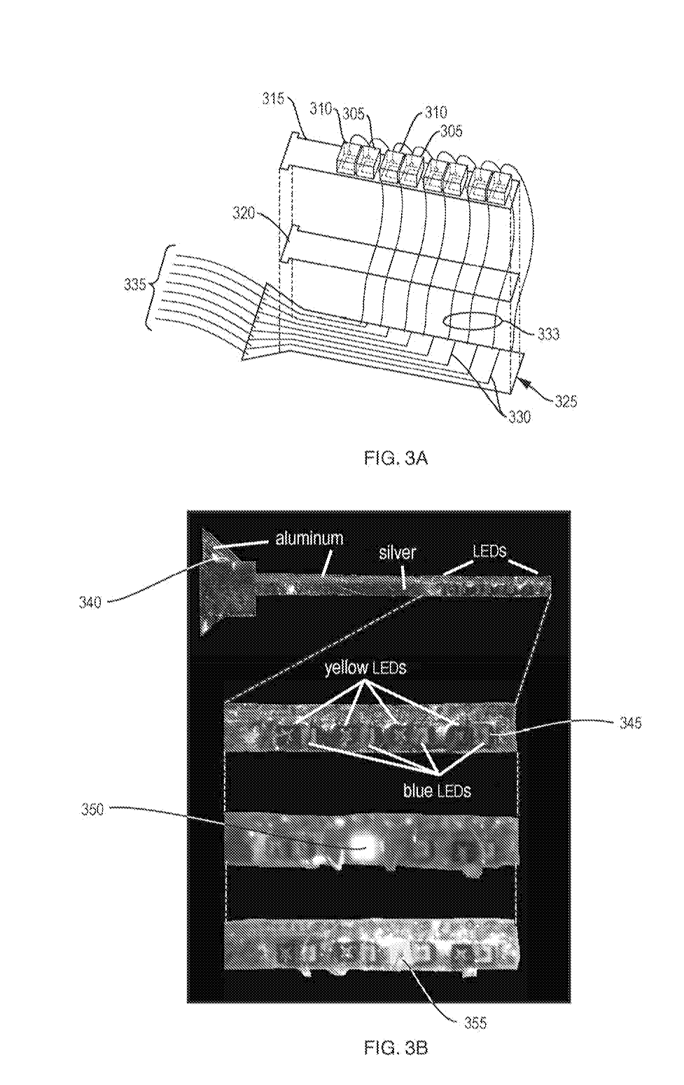

Arranging 1-D probes into a 2-D array is not difficult: almost any plate with appropriate holes will do to organize the 1-D probes into an array. The 1-D probe itself, though, must fit all of the required components into a small enough glass capillary to avoid causing much damage upon insertion into the brain. FIG. 3A depicts an expanded view of an exemplary embodiment of a 1-D probe that is suitable for this purpose and can act as the fundamental building block of the 3-D probe of FIG. 2. In FIG. 3A, blue 305 and yellow 310 LEDs are soldered to ground plane 315. Ground plane 315 attaches, via epoxy layer 320, to underlying base sheet 325. Base sheet 25 has control traces 330 for delivering power to each LED 305, 310 independently via wire bonding 333. Control wires 335 attach each LED control trace 330 to the control circuitry.

In a prototype implementation of the probe of FIG. 3A, 460 nm blue and 590 nm yellow LEDs, 300 microns on a side, were soldered to a 25-micron thick sheet of silver trimmed to be 650 microns wide. The silver sheet acts as the ground plane for the LEDs, and attaches, via a layer of insulating epoxy, to an underlying 25-micron thick aluminum sheet. The aluminum sheet is patterned, using an ultraviolet excimer laser, to have separate control traces for delivering power to each LED independently. Each LED is attached to a unique control trace on the aluminum sheet, via wire bonding.

The entire device is then placed into a thin (<1 mm diameter) glass capillary tube. The ends of the glass capillary are sealed with a biocompatible epoxy, thus sealing in an insulting layer of air between the LED probe and its glass capillary housing. These glass capillaries can then be inserted into holes in a plastic square, resulting in a 2-D array of the 1-D probes. FIG. 3B is a photograph of this prototype implementation. Shown in FIG. 3B, are whole 1-D LED probe 340, a magnified view of LED part 345 of the probe, a magnified view of the LED part of the probe with one blue LED 350 on, and a magnified view of the LED part of the probe with one yellow LED 355 on.

Control traces 330 connect to cable 335 that leads out of the glass capillary, to an multi-channel LED driver circuit, for optimally driving the yellow and blue LEDs. FIG. 3C depicts an exemplary embodiment of LED driver circuit 360 according to this aspect of the present invention. The LED driver circuit 360 comprises typical drive electronics known in the art, such as op-amps 362 and load drivers 363, and is controlled via PIC microcontroller 365. PIC microcontroller 365 talks over USB port 370 to PC 375 running MATLAB or other suitable software known in the art, thus allowing pulse sequences to be uploaded to PIC 365 in real time without necessitating complete reprogramming of the chip.

In a prototype implementation, the fiber arrays were implemented using individual lasers or LEDs, but arrays of vertical cavity surface emitting lasers (VCSELs) or other optical sources may work just as well. If in the future xenon bulbs, halogen lamps, incandescent bulbs, or other light sources become miniaturized enough to fit in, they would also be suitable for use with the present invention (with filters on the bulbs). A particularly appealing way to modulated LED power with a simple circuit is to pulse width modulate (PWM) the LED. A particularly simple wireless method is just to attach an LED to an inductor, which will then be remotely powerable.

In the description and implementation of the present invention, it will be understood by one of ordinary skill in the art that each of the variations on the component parts of the invention are swappable with any of the other variations. Similarly, when a use of the present invention is described with respect to a particular tissue or body part, it will be understood by one of ordinary skill in the art that the invention can be used in a similar manner for other body parts and tissues. For example, if a use is described is for the “brain,” then it may similarly used in any other bodily tissue (e.g., peripheral nerve, pancreas, etc.). As another example, if it is described how to affix something to the skin, it may similarly be used in dealing with muscle and other tissues as well. The terms light source or LED are also used interchangeably, as they all have similar functionality in the context of the present invention. The light produced by the source may be visible light, infrared, spectrally complex, or any other type of light found to be suitable for the particular application. Further, while the use of wireless communications is described herein chiefly in the language of RF transceiving, it will be clear to one of skill in the art that any kind of wireless communication, such as, but not limited to, ultrasound or light may also be advantageously used in the present invention.

Although for central nervous system applications, the 1-D LED probe shown in FIG. 3A is utilized as part of a larger 3-D system, such as the embodiment depicted in FIG. 2, the device shown in FIG. 3A represents in itself an extraordinarily flexible building block for getting light into the nervous system effectively, safely, and inexpensively. In this embodiment, LEDs are simply placed on thin sheets of metal (which can be curved if desired), and the 1-D probes can be made as long or as short as desired, using the rapid-prototyping tools here described. Accordingly, optical cochlear prostheses, vestibular prostheses, and peripheral nerve prostheses may be rapidly deployed using the device of FIGS. 2 and 3A-C. Although the above optical probe is as small or smaller than comparable human devices (e.g., DBS electrodes), even smaller versions capable of controlling neurons in animals as small as a juvenile mouse may be constructed.

In a preferred implementation, an optional dichroic, beamsplitter, or other equivalent optical part known in the art may be attached to a fiber to couple two different light sources (such as, but not limited to, a blue LED and a yellow LED, or a blue laser and a yellow laser) into the fiber, so that the target cells at the end of the fiber can be more easily activated and deactivated by two different colors of light. This may further be generalized to a series of cascaded dichroics, capable of coupling more than two colors of light into the same fiber.

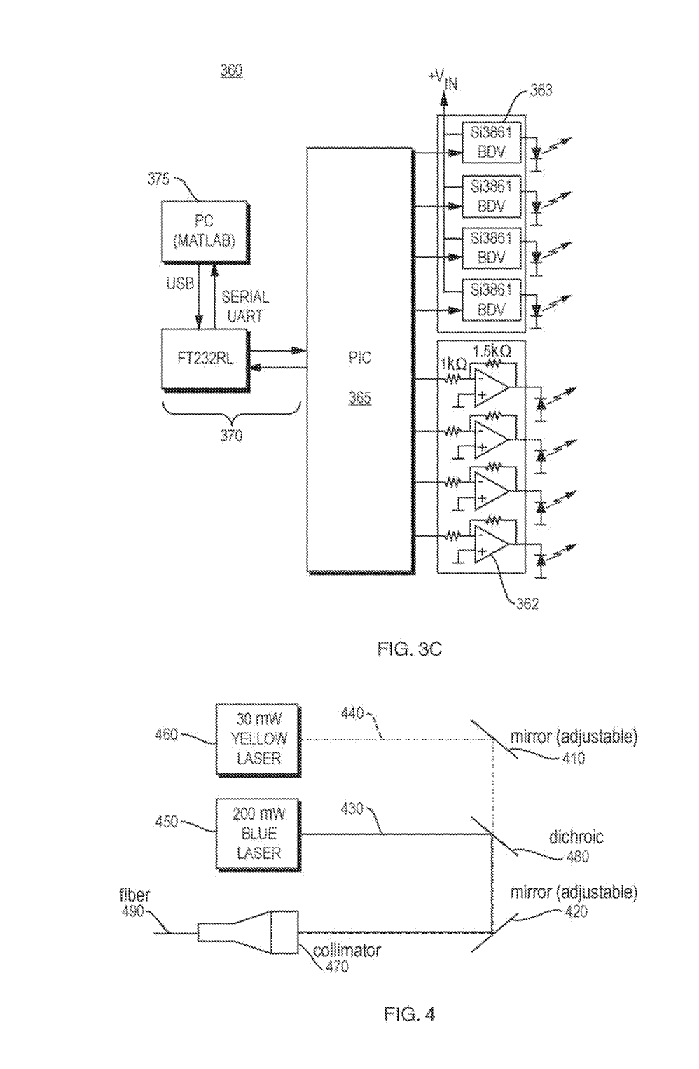

In order to develop a reliable testbed for activation and silencing of genetically-sensitized neurons with blue and yellow light, a prototype system capable of coupling two strong lasers into a single optical fiber was constructed. Two diode-pumped solid state (DPSS) lasers, a yellow (593 nm) 30 mW laser and a blue (473 nm) 200 mW laser (Aixiz Int'l.) were employed. These lasers can be activated for milliseconds at a time when triggered by TTL pulses. FIG. 4 is schematic of an exemplary setup for focusing blue and yellow light down a fiber to be implanted inside the head, for bi-directional control of a single kind of neuron at a single site within the brain, according to one aspect of the present invention.

As shown in FIG. 4, two mirrors 410, 420 on adjustable gimbals steered beams 430, 440 from blue 450 and yellow 460 lasers into fiber collimator 470 (Thorlabs F810SMA), with the assistance of dichroic element 480 (Chroma) that reflects blue light and passes yellow light, thus bringing the two laser beams into collinearity. SMA-terminated multimode fiber 490, 200 microns in diameter and capable of passing light throughout the visible range, was inserted into collimeter 470. The free end of fiber 490, highly polished, could then be inserted into the brain of an experimental animal. In the prototype embodiment, the power coming out of the fiber approached 800 mW/mm² in the blue, sufficient to stimulate ChR2, and 110 mW/mm² in the yellow, sufficient to stimulate Halo. In a mouse, a thin polyimide tube trimmed to the correct length, and inserted into the brain to stereotactically target the brain region of interest, serves easily as a guide cannula for inserting the fiber; gluing a thin washer onto the fiber helped prevent insertion of the fiber into the brain beyond the desired point.

In one embodiment, an optional steerable element, such as, but not limited to, a galvanometer, an acousto-optic deflector, a MEMS mirror, or other steering device is employed on one or both ends of the fiber, to direct light in a controlled way, thus enabling locally selective targeting of the light to specific areas of the tissue, preferably with as few moving parts as possible.

To activate channelrhodopsin-2 and halorhodopsin molecules in mammalian neurons requires light of the appropriate color at a radiant flux of 10 mW/mm² or greater, for maximal activation [E. S. Boyden, F. Zhang, E. Bamberg et al., Nat Neurosci 8 (9), 1263 (2005); X. Han and E. S. Boyden, PLoS ONE 2, e299 (2007); H. Wang, J. Peca, M. Matsuzaki et al., Proc Natl Acad Sci USA 104(19), 8143 (2007)]. A radiant flux of 1 mW/mm² will activate approximately 50% of the molecules, and a radiant flux of 0.1 mW/mm² will activate very few of the molecules [E. S. Boyden, F. Zhang, E. Bamberg et al., Nat Neurosci 8 (9), 1263 (2005); X. Han and E. S. Boyden, PLoS ONE 2, e299 (2007); H. Wang, J. Peca, M. Matsuzaki et al., Proc Natl Acad Sci USA 104 (19), 8143 (2007)]. Since light is absorbed and scattered as it passes through tissue, this means that relatively bright light sources are needed to activate neurons embedded in tissue; furthermore, it implies that for any given light source, there will be heterogeneity in the power that reaches neurons at various distances from the light source.

Simulation of LED light penetration into the brain. The efficacy of optical activation varies across 3 log units of power, from almost no stimulation at 0.1 mW/mm², to near-maximal stimulation at 10 mW/mm². Accordingly, a computational model was implemented that permitted rapidly estimating the power at various distances from blue and yellow LEDs embedded in the brain, aiming for an accuracy level better than a fraction of a log unit. Initially, Monte Carlo simulations of how light emitted from blue and yellow LEDs would be absorbed or scattered in the brain were modeled using 300 μm×300 μm blue (460 nm) and yellow (590 nm) LEDs, placed on the surface of a cube of brain gray matter 4 millimeters on a side. The Monte Carlo simulation was performed by dividing the cube of gray matter into a 200×200×200 grid of voxels, each 20 μm×20 μm×20 μm in dimension. A subset of the models were also tested with 10 μm×10 μm×10 μm voxels, producing identical results and proving that the model resolution was sufficient. Optical modeling and data analysis were done on a PC using MATLAB, and plotted in MATLAB or Excel.

Data was interpolated data from A. N. Yaroslavsky, P. C. Schulze, I. V. Yaroslavsky et al., Physics in medicine and biology 47 (12), 2059 (2002) to obtain scattering coefficients for brain gray matter of 10 mm⁻¹ for blue light and 9 mm⁻¹ for yellow light, and to obtain absorption coefficients of 0.07 mm¹ for blue light and 0.027 mm⁻¹ for yellow light. Since light propagation close to the LED was the factor of interest, before the orientation of photon trajectories is randomized by multiple scattering events, an anisotropic scattering model based upon the Henyey-Greenstein phase function was used, utilizing anisotropy parameters of 0.88 for blue light and 0.89 for yellow light [A. N. Yaroslavsky, P. C. Schulze, I. V. Yaroslavsky et al., Physics in medicine and biology 47 (12), 2059 (2002); T. Binzoni, T. S. Leung, A. H. Gandjbakhche et al., Physics in medicine and biology 51 (17), N313 (2006)]. 10⁶ packets of photons were launched in a Lambertian radiation pattern from random points on the luminous surface of the LED, and their propagation was modeled based on the algorithm of A. N. Yaroslavsky, P. C. Schulze, I. V. Yaroslavsky et al., Physics in medicine and biology 47 (12), 2059 (2002). In essence, whenever a photon packet entered a voxel, the program probabilistically calculated the forecasted traveling distance before the next scattering event. If that traveling distance took the photon packet out of the starting voxel, then the packet was attenuated appropriately for the distance it traveled within the starting voxel, and the process would then restart upon entry of the photon packet into the new voxel. If that traveling distance ended the trip of the photon packet within the starting voxel, then the packet was attenuated appropriately for the distance it traveled within the starting voxel, and a new direction of packet propagation was randomly chosen according to the Henyey-Greenstein function.

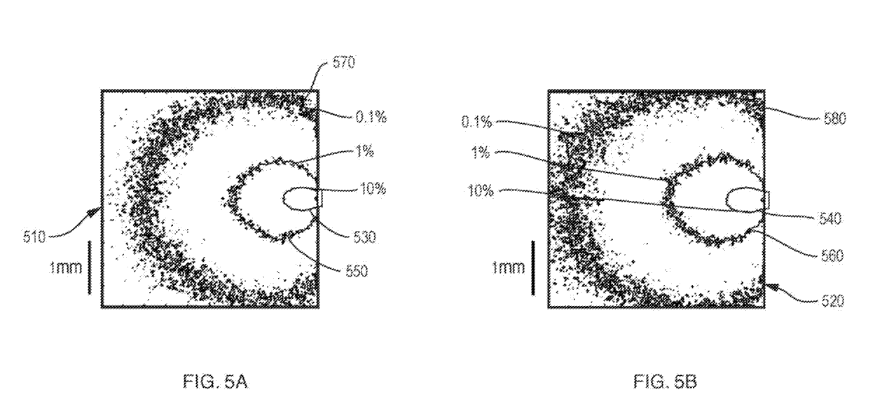

Using this model, FIGS. 5A and 5B, which are Monte Carlo simulations of how blue (FIG. 5A) and yellow (FIG. 5B) photons travel through the brain from LEDs, were generated, depicting the contours at which the light intensity falls off to 10%, 1%, and 0.1% of the intensity of light at the surface of the LED. In FIGS. 5A and B, squares 510, 520 representing 4 mm×4 mm cross-sections taken through the 4 mm×4 mm×4 mm cube are simulated; the cross-sections are taken by slicing the cube through the LED center. Contours represent cross-sections of the surfaces at which the radiant flux drops to 10% 530, 540, 1% 550, 560, and 0.1% 570, 580 of their values on the LED surface.

To implement the model results, the 280 μm×280 μm blue (460 nm) C460EZ290-S2400 LED from Cree [Cree, CPR3CQ.pdf, (2007)] and the 305 μm×305 μm yellow (590 nm) HWFR-B317 LED from Lumileds [Lumileds, DS42.PDF (2007)], were employed. The blue LED has a 1% contour envelope that resembles an ellipse 1.6 mm long and 1.4 mm wide; at a peak power of 24 mW, the 1% contour equates to ˜3 mW/mm² radiant flux—an intensity easily sufficient to activate ChR2. Similarly, for the yellow LED, the 1% contour envelope resembles an ellipse 1.7 mm long and 1.5 mm wide, which at its peak power of 86 mW equates to a radiant flux of ˜15 mW/mm² for yellow light, an intensity sufficient to activate the majority of Halo molecules. Of course, to illuminate smaller volumes, the light power can always be decreased. Thus, the model indicates that standard 300-micron LEDs should be able to illuminate brain volumes across a broad scale, all the way from cubic microns to several cubic millimeters.

The computational model is designed to estimate the power at various distances away from the LED, with accuracy significantly better than a log unit. The quality of the model was assessed experimentally by taking pictures, on a CCD camera, of the blue and yellow LEDs running at 100 microwatts power, through cortical slices of increasing thickness (150, 350, and 550 microns). Experimental measurements were made of the diameter of the contour circle at which the light intensity falls off to 10% of the intensity of light at the surface of the LED. The ratio of this diameter to the diameter of the contour computed via the Monte Carlo model, in a plane a comparable distance away from the LED and parallel to the LED front surface, came out to 0.74±0.33 (mean±standard deviation; n=4 LED-slice thickness combinations), indicating that the model is accurate to the specifications desired. This comparison was repeated for the 1% contours and the ratio was found to be 0.73±0.06 (n=5 LED-slice thickness combinations). There was a trend for the images to be somewhat dimmer than the model would predict. It is possible that vast uncertainty in tissue parameters, which is a subject of ongoing debate in the literature [A. N. Yaroslavsky, P. C. Schulze, I. V. Yaroslavsky et al., Physics in medicine and biology 47 (12), 2059 (2002)], underlies some of this variability. In addition, natural brain heterogeneity may also explain the small differences between the experimentally-obtained data and the simulation model (which assumes purely one kind of gray matter).

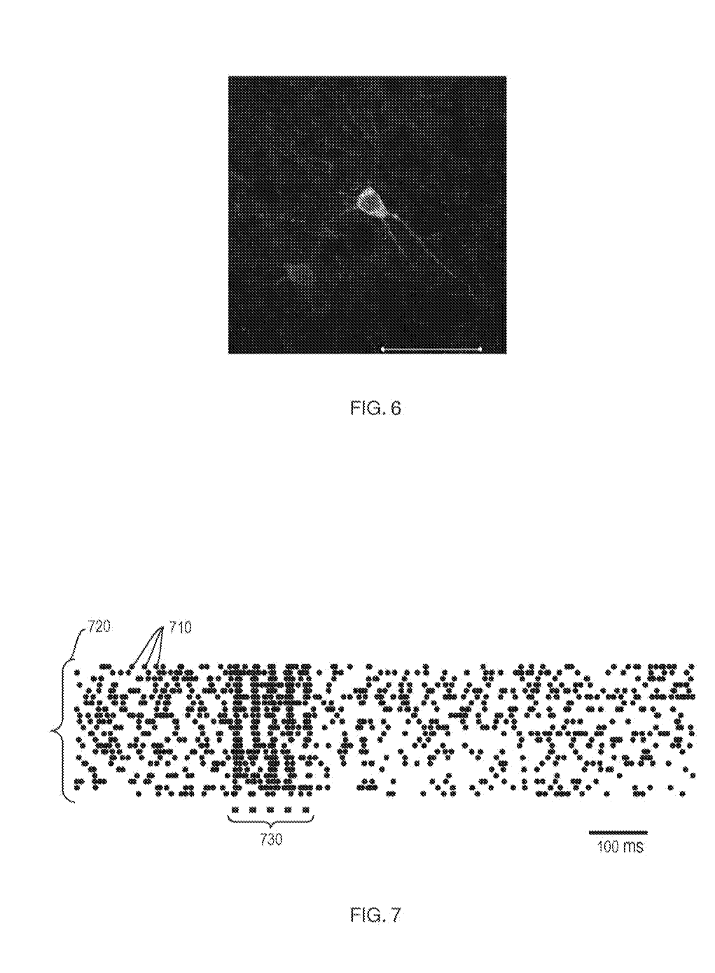

The present invention has been prototyped and experimentally verified using several testing methodologies. The ability to make specific neurons in the brain light-sensitive, using a viral approach and control of neural activity in the cortex of the non-human primate, a key step in the translation of such technology for human clinical use, were demonstrated. In these tests, one experiment assessed intact tissue expression in mouse. Mammalian codon-optimized forms of channelrhodopsn-2 and halorhodopsin, abbreviated as hChR2 and Halo, have been previously developed [X. Han and E. S. Boyden, PLoS ONE 2, e299 (2007)]. These genes were inserted into a lentiviral vector that allows cloning in different promoters, or DNA regulatory elements, upstream of the gene of interest. For example, the CaMKII promoter targets predominantly excitatory neurons. FIG. 6 is a photograph of neurons in the mouse brain expressing Halo-GFP under the CaMKII promoter, which preferentially labels excitatory neurons [T. Dittgen, A. Nimmerjahn, S. Komai et al., Proc Natl Acad Sci USA 101 (52), 18206 (2004)]. In FIG. 6, the Scalebar represents 50 μm. A pipeline was developed for obtaining promoters by cloning them out of bacterial artificial chromosomes (BACs) and then inserting them into lentiviral plasmids upstream of hChR2 or Halo. Small virus test batches are then created, and promoter strength and selectivity in the mouse brain are subsequently rapidly screened. By screening promoters in a wholesale fashion, it is possible to identify and validate new candidate promoters for targeting specific cell types.

For this experiment, replication incompetent lentiviruses were produced via triple transfection of plasmids containing the promoter and gene of interest (e.g., F(CK)-Halo-GFP or F(CK)-ChR2-GFP), the viral helper plasmid (pΔ8.91), and the pseudotyping plasmid (pMD2.G, encoding the coat protein VSV-G). Briefly, HEK293FT cells (Invitrogen) were plated onto four T175 flasks in D10 medium (comprising DMEM+10% FBS+1% pen-strep, 1% sodium pyruvate, and 1% sodium bicarbonate). At 100% confluence, cells were transfected with DNA using Fugene: 22 micrograms of plasmids containing the promoter and gene of interest, 15 micrograms of pΔ8.91, and 5 micrograms of pMD2.G, were mixed with 132 microliters of Fugene 6 and 4.5 mL of MEM, prepared according to the instructions of the manufacturers of Fugene. 24 hours later, the cells were washed with D10 and then given 30 mL of virus production media (comprising Opti-MEM w/GlutaMAX-I+1% pen-strep, 1% sodium pyruvate, and 1% sodium bicarbonate). 48 hours later, the supernatant was harvested, filtered through a 0.45 micron filter flask (pre-wetted with D10), and then the filtrate was ultracentrifuged over a 20% sucrose cushion at 22000 rpm in a SW-28 rotor for 2 hours at 4° C. The pellet was then resuspended in 30 microliters of PBS over a period of several hours, and aliquoted the virus for storage at −80° C.

Viruses were tested for efficacy in sensitizing specific neuron types to being activated/silenced by light by being injected into the cerebral cortex of mice. Mice were anesthetized with 1.25-2% isoflurane and placed into a custom stereotax. A dental drill was used to make a small craniotomy, through which 1-2 microliters of virus was injected into the cerebral cortex of the mouse brain. The virus was injected through a pulled borosilicate glass pipette (tip ˜5 microns wide; shank ˜4 mm long), pulled with a Sutter P-97 puller. This glass pipette was connected to a Hamilton syringe (placed in a syringe pump, from Harvard Apparatus) via a thin plastic tube filled with silicone oil. Virus infusion was carried out by actuating the Harvard Apparatus pump to inject slowly (e.g., 0.1 microliters per minute) over a period of 20 minutes. After the viral payload was delivered, 10-20 minutes was allowed to pass in order for the virus to diffuse away from the site of injection before withdrawing the pipette at a slow rate (e.g., 2 mm/min). The scalp of the mouse was then sealed with Vetbond, and the animal administered buprenorphine and returned to its home cage.

One to four weeks after virus injection, acute slices of brain tissue were cut in order to assess the targeted cells for strength and specificity of gene expression, as described in J. Bischofberger, D. Engel, L. Li et al., Nature protocols 1 (4), 2075 (2006). Briefly, mice were anesthetized with isoflurane and decapitated, and the brains were removed to ice cold cutting solution (87 mM NaCl, 25 mM NaHCO₃, 25 mM glucose, 75 mM sucrose, 2.5 mM KCl, 1.25 mM NaH₂PO₄, 0.5 mM CaCl₂ and 7 mM MgCl₂, bubbled with 95% O₂/5% CO₂). Time between death and completion of brain removal was less than 1 minute. Brains were then blocked and glued to a dish for cutting with a vibrating tissue slicer (Leica VT1000S) into sections 240 microns thick. Slices were incubated at 35° C. for 30 minutes, then stored at room temperature. Slices were acutely examined in physiological saline (125 mM NaCl 25 mM NaHCO₃, 25 mM glucose, 2.5 mM KCl, 1.25 mM NaH₂PO₄, 2 mM CaCl₂, and 1 mM MgCl₂). At this time, cells were examined electrophysiologically through whole-cell patch clamp (access resistance, 4-8 megaohms), conducted with a Molecular Devices Multiclamp 700B setup.

In a second test, optically-elicited spike trains were obtained from a primate cortex. In order to translate the precise optical neural control technology of the present invention to humans, the feasibility and safety must be tested in non-human primates, whose brains and other bodily systems more closely resemble humans than do those of rodents. Accordingly, both for safety and efficacy testing, and for animal model development, the primate may represent a natural pre-clinical testbed for optical neural control technologies.

The basic methods employed in the test were as described in T. Womelsdorf, P. Fries, P. P. Mitra et al., Nature 439 (7077), 733 (2006). Briefly, a craniotomy and plastic chamber implantation was performed over the frontal eye field (FEF) region of the cortex of a rhesus macaque that had been prepared with a headpost, and trained to fixate upon a point. Recordings were made with tungsten microelectrodes (1-2 megaohm impedance) that were slowly advanced through cortical tissue until single units were isolated, acquiring data via a Multichannel Acquisition Processor from Plexon, Inc. Spike recordings were filtered, and spikes isolated using an interactively-set threshold. To facilitate precise and repeatable optical stimulation and recording, a recording chamber insert with a grid of evenly-spaced holes was designed and fabricated, in order to facilitate optimal placement of electrodes and optical fibers.

As a test case, a male rhesus macaque was prepared for cortical recording in the frontal eye field (FEF) area of cortex, as predicted by MRI scans and validated with electrophysiology. Surgically injection of 1-4 μL of virus expressing ChR2-GFP under the CaMKII promoter into a site within FEF was undertaken, using the equipment described previously. After a 2-week waiting period for the virus to express, and a polished, 200 micron-thick optical fiber whose other end was fiber-coupled to a 200 mW blue (473 nm) laser was inserted. After optimizing the recording, the laser was activated to fire brief pulses of blue light by delivering 10 ms-long TTL pulses separated by 20 ms pauses (i.e., 33.3 Hz stimulation), and elicited trains of well-timed spikes.

FIG. 7 is a raster plot showing the experimentally-obtained occurrences of spikes elicited from an excitatory neuron expressing ChR2 in a monkey cortex in response to a brief train of blue light stimulation. As shown in FIG. 7, spikes (black dots) 710, in 22 consecutive spike trains (each row of black dots) 720 were elicited in response to blue light stimulation. Each horizontal row 720 reflects one recording of the response to five blue pulses 730 of light, each lasting 10 ms and separated from the next by 20 ms (i.e., 33.3 Hz stimulation rate). This result demonstrates, for the first time, that both the genetic targeting of neurons, and the use of light to activate them, can work successfully in the macaque cortex, a key translational milestone along the road to human use of the present invention.

The present invention is advantageously employed for the purpose of activating or silencing circuits in a targeted way. The device is capable of activating and silencing neurons at the scale of microns to millimeters, and is inexpensive, reliable, and easy to use. Effective viral targeting of excitatory neurons in the intact brain has been demonstrated using the present invention. The experimental results have demonstrated for the first time that optical neural control technologies are capable of working in the intact primate brain, a major milestone in the quest to enable novel human therapies. The present invention may be advantageously used to understand how to control neural circuits to compensate for the loss or alteration of specific cell types, as often occur in neurological and psychiatric diseases ranging from obesity, to pain, to Parkinson's, to epilepsy. Neural control technologies such as DBS and TMS are increasing in popularity for the treatment of a great number of diseases, but because their mechanisms of therapeutic action are poorly understood, few generalized strategies or principles have emerged governing the design of treatments that use these technologies. By explicitly seeking out neural targets and activity patterns that enable control of neural circuits, a better understanding may be obtained of the principles governing the correction of deficits at the neural-circuit level.

While the present invention is applied herein to use light to excite and inhibit electrically excitable cells; it will be clear to one of skill in the art that it may be adapted to deliver light to other realms, such as, but not limited to, to drive the production of cAMP in deep tissue, to simulate the action of a G-protein coupled receptor acting drug, or to change the pH of a cell. There are many therapeutic reasons to desire these abilities. The present invention may be advantageously adapted to enable cell- and circuit-level control in human patients of neurological and psychiatric disorders. Over 600 patients have been treated with genes delivered into their cells via the adeno-associated virus (AAV) vector, in 48 separate trials, without a single serious adverse event resulting from the virus [Nat Biotechnol 25 (9), 949 (2007)]. AAV is ubiquitous (perhaps 90% of people have been exposed to it) and causes no symptoms by itself. Accordingly, the field of gene therapy is moving in directions that could make optical prosthetics highly beneficial and practical at some point in the not-so-distant future. Furthermore, the fact that ChR2 and Halo may be useful for controlling many neural circuits throughout the nervous system may simplify the exploration of this gene therapy space: once these molecules have been tested for basic efficacy and safety in a few kinds of neuron, subsequent validation attempts may proceed quite rapidly, for use in novel cell types. Systems for optical targeting of specific neural circuit elements may enable a new generation of high-precision therapies for brain disorders.

While a preferred embodiment of the present invention is disclosed, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

What is claimed is:

1. A prosthetic device for optical control of target cells, comprising:
a probe, the probe comprising:
a plurality of light sources;
a plurality of drive circuit connections, at least one drive circuit connection being connected to each light source; and
a housing surrounding the plurality of light sources and the drive circuit connections; and
drive circuitry for driving and controlling the probe, the drive circuitry being capable of communicating with the probe through the drive circuit connections.

2. The device of claim 1, the drive circuit connections and drive circuitry further comprising circuitry for wireless communication.

3. The device of claim 1, wherein the light sources are light-emitting diodes.

4. The device of claim 3, wherein the light-emitting diodes are blue and yellow.

5. The device of claim 1, wherein the light sources are lasers.

6. The device of claim 1, wherein the housing is a glass capillary tube.

7. The device of claim 6, wherein the plurality of light sources is assembled into an array.

8. The device of claim 7, wherein the light source array is linear.

9. The device of claim 1, the prosthetic device further comprising sensors for monitoring the target cells.

10. An array comprising prosthetic devices according to claim 1.

11. A prosthetic device for optical control of target cells, comprising:
an array of probes, each probe comprising:
a plurality of light sources;
a plurality of drive circuit connections, at least one drive circuit connection being connected to each light source; and
a housing surrounding the plurality of light sources and the drive circuit connections;
supporting hardware that holds the probes in position with respect to each other and the target cells; and
drive circuitry for driving and controlling the probes, the drive circuitry being capable of communicating with the probes through the drive circuit connections

12. The device of claim 11, the drive circuit connections and drive circuitry further comprising circuitry for wireless communication.

13. The device of claim 11, wherein the light sources are light-emitting diodes.

14. The device of claim 13, wherein the light-emitting diodes are blue and yellow.

15. The device of claim 11, wherein the light sources are lasers.

16. The device of claim 11, wherein the housing is a glass capillary tube.

17. The device of claim 16, wherein the plurality of light sources is assembled into an array.

18. The device of claim 17, wherein the light source array is linear.

19. The device of claim 11, the prosthetic device further comprising sensors for monitoring the target cells.

20. The device of claim 11, wherein the array of probes is 3-dimensional.
